(19) United States
(12) Patent Application Publication
(10) Pub. No.: US 2013/0096362 A1
(43) Pub. Date: Apr. 18, 2013

(54) CONFIGURABLE MAGNETIC ORIENTATION SYSTEM

(71) Applicant: David Balzer, Orange, CA (US)

(72) Inventor: David Balzer, Orange, CA (US)

(21) Appl. No.: 13/544,950

(22) Filed: Jul. 9, 2012

Related U.S. Application Data
(60) Provisional application No. 61/547,218, filed on Oct. 14, 2011.

(51) Int. Cl. A61N 2/08; B23P 17/04

(52) U.S. Cl. 600/9; 29/428

(57) ABSTRACT

A system for providing at an application surface of a therapeutic device a multitude of magnetic flux lines of different angles and strengths, including a plurality of arms and a plurality of magnets. Each arm piece may have one or more connecting members and one or more magnet receiving sockets or recesses. At least some of the one or more connecting members are generally configured to attach to one or more attachment or mating mechanisms on other arms. The magnets are placed in the sockets, and the connecting members of the plurality of arms are connected such that the arms form huh-and-spoke configurations, chain-configurations, loops, lines, or any combination of the above.

### CROSS-REFERENCES TO RELATED APPLICATIONS

This patent application claims the benefit of U.S. Provisional Application No. 61/547,218, filed Oct. 14, 2011, which application is incorporated herein by this reference thereto.

### BACKGROUND OF THE INVENTION

1. Field of the Invention

This invention relates to the field of magnetic therapy devices, and in particular to the field of consumer magnetic therapy devices that produce a multitude of magnetic flux lines at the application surface.

2. Description of the Related Art

There are a number of magnetic therapy devices that produce a multitude of magnetic flux lines at the application surface. Most, however, involve costly manufacturing processes and result in manufacturing processes that are relatively rigid in the resultant magnetic configuration.

### BRIEF SUMMARY OF THE INVENTION

One embodiment of the present invention comprises a system for providing at an application surface of a therapeutic device a multitude of magnetic flux lines of different angles and strengths, including a plurality of arms and a plurality of magnets. Each arm piece may have one or more attachment mechanisms or connecting members and one or more magnet receiving sockets. At least some of the one or more connecting members are generally configured to attach to one or more attachments or mating mechanisms on other arms. The magnets are placed in the sockets, and the connecting members of the plurality of arms are connected such that the arms form hub-and-spoke configurations, chain-configurations, loops, lines, or any combination of the above.

In some embodiments, the sockets may also have one or more magnet retaining surfaces and one or more magnet orientation members. The magnets are then placed in the sockets and held in place by the retaining surface. At least some of the magnets are also held pre-determined orientations by the orientation members in the sockets.

### BRIEF DESCRIPTION THE DRAWINGS

FIG. 1 is a perspective view of two arms connected together and retaining a total of five magnets in keeping with one embodiment of the present invention with a square around one of the arms.

FIG. 2 is a side view of the one arms highlighted in FIG. 1.

FIG. 3 is a perspective view of the therapeutic device of FIG. 1 with a square around the other arm.

FIG. 4 is a side view of the one arm highlighted in FIG. 3.

FIG. 5 is a top plan view of another two arm embodiment in keeping with the present invention.

FIG. 6 is a side view of one of the arms of the therapeutic device shown in FIG. 5.

FIG. 7A is a top plan view of a magnet in which its magnetic north is oriented perpendicular to the plane of the paper in keeping with one embodiment of the present invention.

FIG. 7B is a top plan view of a magnet in which its magnetic north is oriented roughly 45E off the normal axis of FIG. 7A toward the viewer.

FIG. 7C is a top plan view of a magnet in which its magnetic north is oriented roughly 45E to the right of the normal axis of FIG. 7A.

FIG. 7D is a top plan view of a magnet in which its magnetic north is oriented roughly 45E off the normal axis of FIG. 7A away from the viewer.

FIG. 7E is a top plan view of a magnet in which its magnetic north is oriented roughly 45E to the left of the normal axis of FIG. 7A.

FIG. 8 is a top plan view of three arms connected together in a snowflake configuration retaining a total of seven magnets in keeping with one embodiment of the present invention.

FIG. 9 is a top plan view of three arms connected together in a triangle configuration retaining a total of six magnets keeping with one embodiment of the present invention.

FIG. 10 is a top plan view of six arms connected together in a square or diamond lattice configuration retaining a total of nine magnets in keeping with one embodiment of the present invention.

### DESCRIPTION OF THE PREFERRED EMBODIMENT(S)

The detailed description set forth below connection with the appended drawings is intended as a description of presently-preferred embodiments of the invention and is not intended to represent the only forms in which the present invention may be constructed and/or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

As shown FIGS. 1 through 6, one embodiment of the present invention comprises a spoke configuration to position and orient two or more magnets. Each spoke or arm 20 comprises one or more magnet receiving sockets or zones 22, with each receiving zone 22 holding a magnet 30 in a pre-determined orientation. The arms or spokes 20 can be cast or is injection molded from any number of common plastic materials prior to adding the magnets 30.

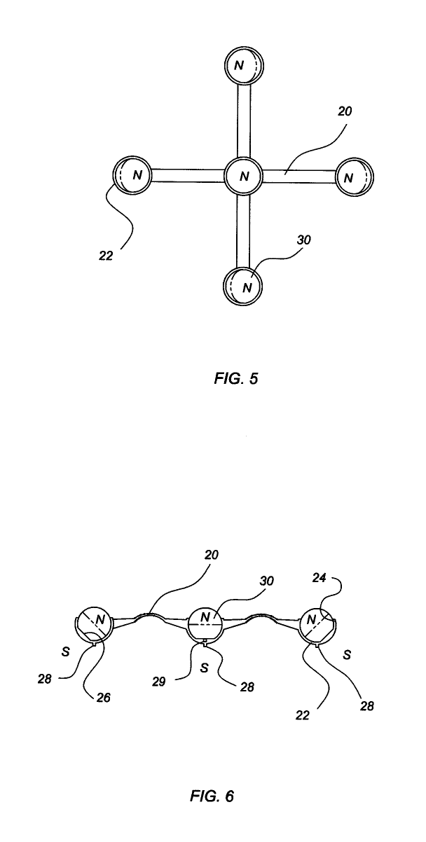

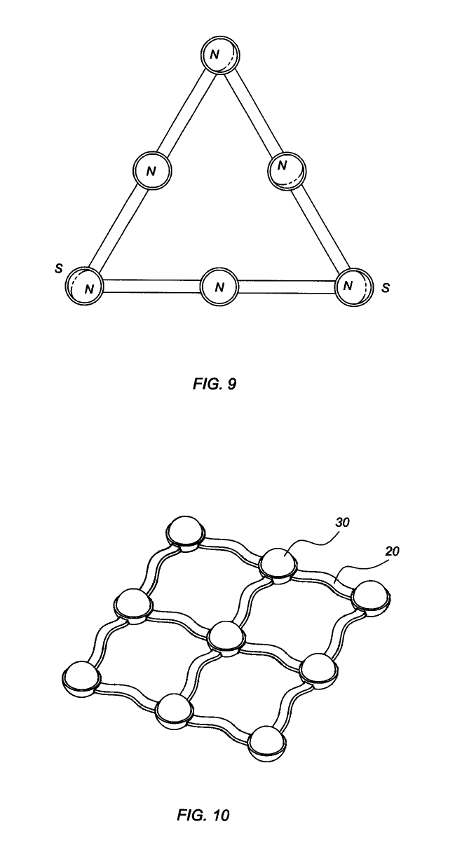

During the assembly process, each magnet 30 can be inserted into its receiving orifice 22 and locked into place. Each spoke or arm 20 comprises one or more connecting members 28 and 29 to connect one arm) to another. The attachment or connecting mechanisms or embers 28 may be on the bottom of the middle portion of the arm 20 or on the bottom of one or both of the end portions of arm 20, or they may be on both portions of arm 20, as shown in FIG. 6. As a result, the arms 20 may connect to form a hub-and-spoke configuration as shown FIGS. 5 and 8 or a chain-link configuration as shown in FIGS. 9 and 10. The resulting assembly will produce a magnetic therapeutic device 10 that can be any size or shape and incorporate any number of magnets with a myriad of orientations.

The attachment members 28 shown in FIG. 6 are ridges designed to mate with mating attachment members 29, which is illustrated as a notch in FIG. 6. The attachment members could, however, be any number of other such attaching configurations, including without limitation, mating edge features, a hole and post configuration, a two-hole configuration in which both adjacent arms have a recess or hole into which a bearing or pivot ball or bar is mutually placed, or simple adhesives pressed between two flattened or mating surfaces on two adjacent arms 20 where the two arm 20 cross over one another. The attaching features may be fixedly attached or they may be rotatably or detachably attached so that the larger structure retains additional when in use or between uses.

Each socket or recess 22 comprises a retaining surface or feature 24 to hold the magnet in the socket 22. In FIG. 1, the retaining surface 24 is an upper edge or lip of socket 22, but it could alternatively or additionally comprise a flange, ridge, inward facing nubs or fingers, or the like. One purpose of the retaining surface 24 in such embodiments is to retain the magnet 30 within, or at least partially within, the receiving recess or zone 22 once it is placed therein during assembly.

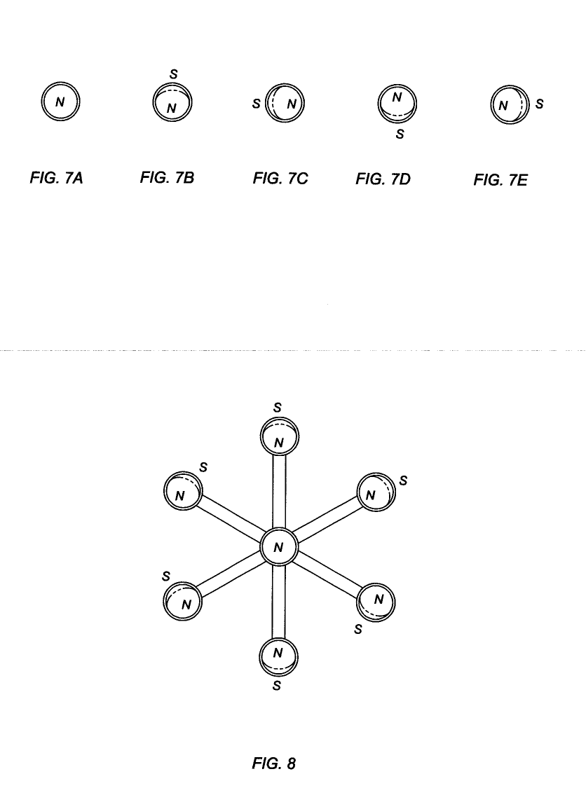

Additionally, each socket may also comprise an orientation member 26 to engage with a mating surface member 36 on the magnet 30 and hold the magnet 30 in a predetermined orientation. The embodiment illustrated in FIGS. 7A to 7E provides for five different possible locations for orientation member 26, numbered for the convenience of this discussion, 0 through 4. In each instance in FIGS. 7A to 7E, the orientation member 26 is illustrated as a flattened portion of the otherwise concave inner surface of socket 22 that mates with a flattened portion on an otherwise spherical magnet 30. The orientation member 26 could, however, be a small post, protrusion, or other keying structure that mates with a small indentation or other matching key structure on magnet 30. The orientation member 26 could instead be the result of the shape of magnet 30 itself, such as an elongated axis of a receiving zone or recess 22 that is rectangular configured to receive a rectangular magnet 30, a wall or notch in a donut-shaped recess for receiving a toroidal or donut-shaped magnet having a thin gap or the like in its body, etc. The purpose of the orientation member 26, therefore, in such embodiments is to make sure that magnet 30 is held within socket 22 at a particular predetermined orientation so that the magnetic flux lines 32 of the magnet 30 are likewise maintained at a particular predetermined orientation once placed in the socket 22.

The magnets 30 may all be identical in shape and strength, or they may comprise a variety of shapes or strengths. The embodiment illustrated in FIGS. 1 through 7E comprise magnets 30 that are spherical with one flattened edge, but could be any number of other shapes, including rectangular, flat, cylindrical, etc. One benefit from using spherical magnets is that the magnetic flux lines and the orientation surface may easily be oriented at any angle with respect to one another. Benefits of any other shape for the magnets are evident, such as clearance concerns for flat or thin magnet, close packing opportunities for rectangular or hexagonal magnets, etc.

Combined with the embodiment of FIG. 1, which illustrates an arm 20 having three sockets along its length, these five possible locations for orientation member 26 can produce 75 perturbations, namely, 0-0-0 through 4-4-4, excluding those combinations that are mere mirror ages of another combination:

TABLE 1
The 75 alternate perturbations of a single arm 20

| Left | Middle | Right |
|---|---|---|
| 0 | 0 | 0 |
| 0 | 0 | 1 |
| 0 | 0 | 2 |
| 0 | 0 | 3 |
| 0 | 0 | 4 |
| 0 | 1 | 0 |
| 0 | 1 | 1 |
| 0 | 1 | 2 |
| 0 | 1 | 3 |
| 0 | 1 | 4 |
| 0 | 2 | 0 |
| 0 | 2 | 1 |
| 0 | 2 | 2 |
| 0 | 2 | 3 |
| 0 | 2 | 4 |
| 0 | 3 | 0 |
| 0 | 3 | 1 |
| 0 | 3 | 2 |
| 0 | 3 | 3 |
| 0 | 3 | 4 |
| 0 | 4 | 0 |
| 0 | 4 | 1 |
| 0 | 4 | 2 |
| 0 | 4 | 3 |
| 0 | 4 | 4 |
| 1 | 0 | 1 |
| 1 | 0 | 2 |
| 1 | 0 | 3 |
| 1 | 0 | 4 |
| 1 | 1 | 1 |
| 1 | 1 | 2 |
| 1 | 1 | 3 |
| 1 | 1 | 4 |
| 1 | 2 | 1 |
| 1 | 2 | 2 |
| 1 | 2 | 3 |
| 1 | 2 | 4 |
| 1 | 3 | 1 |
| 1 | 3 | 2 |
| 1 | 3 | 3 |
| 1 | 3 | 4 |
| 1 | 4 | 1 |
| 1 | 4 | 2 |
| 1 | 4 | 3 |
| 1 | 4 | 4 |
| 2 | 0 | 2 |
| 2 | 0 | 3 |
| 2 | 0 | 4 |
| 2 | 1 | 2 |
| 2 | 1 | 3 |
| 2 | 1 | 4 |
| 2 | 2 | 2 |
| 2 | 2 | 3 |
| 2 | 2 | 4 |
| 2 | 3 | 2 |
| 2 | 3 | 3 |
| 2 | 3 | 4 |
| 2 | 4 | 2 |
| 2 | 4 | 3 |
| 2 | 4 | 4 |
| 3 | 0 | 3 |
| 3 | 0 | 4 |
| 3 | 1 | 3 |
| 3 | 1 | 4 |
| 3 | 2 | 3 |
| 3 | 2 | 4 |
| 3 | 3 | 3 |
| 3 | 3 | 4 |
| 3 | 4 | 3 |
| 3 | 4 | 4 |
| 4 | 0 | 4 |
| 4 | 1 | 4 |
| 4 | 2 | 4 |
| 4 | 3 | 4 |
| 4 | 4 | 4 |

For a two-arm hub-and-spoke configuration, in which the spokes lie along the diagonals of a square, these perturbations combine to produce 750 possible combinations. A three-arm hub-and-spoke configuration, in which the spokes lie along the diagonals of hexagon, combines to produce 7500 possible combinations.

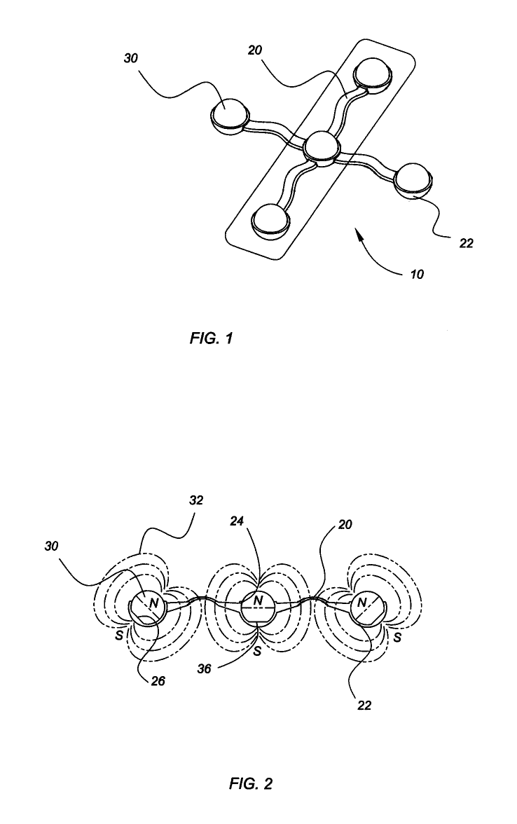

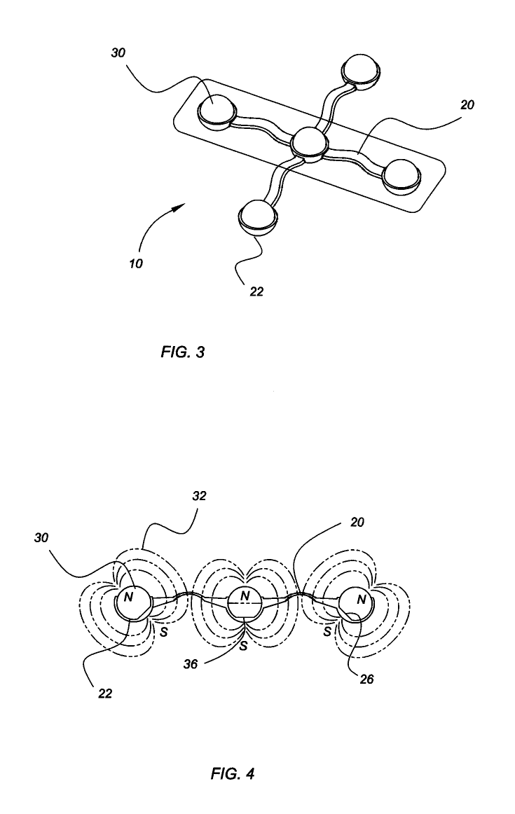

Another embodiment of the present invention also encompasses configurations of three or lore such arms 20 connected together in chain-like loop or ring configurations, as shown in FIGS. 7A through 7B. As with the hub-and-spoke configurations, the number of perturbations grows quickly. For a simple triangle configuration, in which there are three three-socket arms 20 shown FIGS. 2, 4, and 6, each having one of the orientation members shown FIGS. 3A through 3E, and believed to be combinable to produce 3750 different possible configurations. A square ring configuration, in which four arms 20 lie along the perimeter of a square, combines to produce 37,500 possible different combinations, and a diamond configuration, having four arms 20 around the perimeter and a fifth arm 20 connecting two of the opposing corners of the diamond, combines to produce 187,500 possible combinations. The lattice configuration shown in FIG. 10 comprises six arms 20 interconnected to produce over 400,000 possible combinations.

This great diversity arises from only one particularly simple arm 20 configuration, namely, an arm 20 having three sockets 22, each exhibiting an orientation surface 26 chosen from five standard, orthogonal locations within the socket 22 shown FIGS. 7A through 7E. This configuration is conducive to programming the die cast or injection molding machinery to produce any series of arms 20 having first, second, and third sockets of any predetermined orientation so that the assembly process may be extensively automated. For example, a fabric blanket could require sixty such magnetic therapy devices 10, in an array of 10×6, each device 10 having two arms 20—the first arm 20 having an orientation member 26 pattern of 1-0-3, and the second arm 20 having an orientation member 26 pattern of 2-0-4, as shown in FIG. 1. The injection molding machine could either produce sixty 1-0-3 arms and sixty 2-0-4 arms or sixty 1-0-3 arm/2-0-4 arm pairs, depending on the downstream assembly requirements.

Moreover, the arms 20 need not be limited to the three-socket arm 20 illustrated in FIGS. 1 and 2, and the orientation members 26 need not be limited to the relatively simple five orthogonal orientations illustrated FIGS. 3A through 3E. The present invention equally envisions a vast number of alternative arms members, including ones with more sockets, non-linear arms, arrays, and the like. The sockets themselves may instead be recesses, orifices, indentations, pockets, etc. and they may have numerous alternatives when it comes to the retaining surfaces, such as snaps, belts, clasps, flanges, ridges, inward facing detents or fingers, etc., and when it comes to the orientation members, such as nubs, posts, guide wires, etc. The orientation members 26 could be positioned among a vastly greater number of possible locations, including dividing up the magnet receiving zone into thirteen sectors instead of five. The magnets 30 themselves could instead be magnetized in various orientations or even randomly about or around the mating feature 36.

Thus, one purpose of some embodiments of the present invention is to have a system of interconnecting basic units that produce a quick, convenient, and robust combination of arms, connectors, and receiving zones having one or more retaining surfaces, and optionally also having one or more magnet orientation surfaces or members. The result is a programmable system that can produce numerous base units, namely, the combination of an arm 20 holding one or more magnets 30, which when combined with other such base units may quickly produce a large variety of magnetic therapy devices 10 having predetermined and relatively complex patterns of magnetic flux lines 32 at an application surface.

One such purpose is the flexibility of how basic units, such as these arms 20, can hold individual magnets 30 at pre-determined orientations and can attach together with other arms 20 that also hold individual magnets 30 at pre-determined orientations to provide a richly diverse pattern of surface magnetic flux lines 32—both in terms of orientation and in terms of strength and depth—to apply to a user's body. Alternatively, some embodiments encompass arms 20 that hold magnets 30 at wholly or predominantly random magnetic orientations, which may provide a richly diverse multitude of surface magnetic flux lines 32 and strengths to apply to a user's body.

In some embodiments, the magnetic configuration of the present invention also comprises two or more arms that can be pressed together, or otherwise connected, at their centers or medial portions (to form spoke-like or snowflake-like patterns as shown in FIGS. 1, 3, 5, and 8) or at their end portions (to form chain-like patterns as shown FIG. 9), with each arm 20 grasping one or snore individual magnets 30 in pre-determined orientations to provide a richly diverse pattern of surface magnetic flux lines 32 to apply to a user's body and that the manufacturer can readily change (in the next batch of product) the pattern of surface magnetic flux lines using a plurality of the same basic unit (the arms 20) should research suggest an alternative configuration of magnetic flux lines 32 is better.

Additionally, the arms 20 may be bowed in their medial portions as illustrated in FIGS. 1 and 6. This will help reduce the general waviness of the front face of the device so that it feels somewhat flatter to the user at the application surface. The arm 20, and in particular to medial portion, may be rigid. The arms 20, however, may instead be flexible so that the application surface can flex when the user, through his or her own weight or other forces, forces the application surface to bend. One effect of using flexible arms, therefore, is that the magnetic flux lines may remain as close as possible to the target area of the user's body. Another beneficial effect is that the magnetic flux lines could be moved dynamically during use and interact with the magnetic flux lines of neighboring magnets along the application surface so that the target area of the user's body may encounter a dynamically changing set of magnetic flux lines over time.

While the present invention has been described with regards to particular embodiments, it is recognized that additional variations of the present invention may be devised without departing from the inventive concept.

What is claimed is:

1. A system for providing at an application surface of a therapeutic device a multitude of magnetic flux lines of different strengths and angular orientation, comprising:
a plurality of magnets, and
a plurality of rigid arms for spacing the plurality of magnets at predetermined distances from one another, said arm piece comprising
a first end portion,
a second end portion opposing said first end portion,
a medial portion between the first and second end portions to space the first and second end portions a predetermined distance from one another, said medial portion being bowed to provide support to the user in between each individual magnet,
one or more connecting members configured to attach to a like or mating attachment mechanism of an adjacent arm piece, and
one or more magnet receiving recesses located on the first and second end portions of the arms, each magnet receiving recess having at least one magnet retaining surface and at least one magnet orientation member, said magnet orientation member comprising a flattened surface on the inside of the recess such that, when used in combination with the magnet retaining surface, the magnet orientation member properly orients the magnet in a predetermined angular orientation,
wherein the plurality of magnets are retained in the magnet receiving recesses by said retaining surfaces, held in relation to one another and spaced apart at predetermined distances from one another by the plurality of arms, and held in predetermined orientations by said orientation members, thereby providing at an application surface of a therapeutic device a multitude of magnetic flux lines of different strengths and angular orientations.

2. The system as in claim 1, wherein the one or more attachment members are located on the medial portions of the arms to produce snowflake-like patterns by connecting together two or more arms.

3. A system for providing at an application surface of a therapeutic device a multitude of magnetic flux lines of different strengths and angular orientation, comprising:
a plurality of magnets, and
a plurality of arms for spacing the plurality of magnets at predetermined distances from one another, said arm piece comprising
a first end portion,
a second end portion opposing said first end portion,
a medial portion between the first and second end portions to space the first and second end portions a predetermined distance from one another,
one or more connecting members configured to attach to a like or mating attachment mechanism of an adjacent arm piece, and
one or more magnet receiving recesses each having at least one magnet retaining surface and at least one magnet orientation member,
wherein the plurality of magnets are retained in the magnet receiving recesses by said retaining surfaces, held in relation to one another and spaced apart at predetermined distances from one another by the plurality of arms, and held in predetermined orientations by said orientation members, thereby providing at an application surface of a therapeutic device a multitude of magnetic flux lines of different strengths and angular orientations.

4. The system as in claim 1, wherein the one or more attachment members are located on the medial portions of the arms to produce snowflake-like patterns by connecting together two or more arms.

5. The system as in claim 1, wherein the one or more attachment members are located on the first and second end portions of the arms to produce chain-like patterns by connecting together two or more arms end to end.

6. The system as in claim 1, wherein the arms are rigid.

7. The system as in claim 1, wherein at least some of the arms are flexible so that they may flex when used thereby causing the application surface to bend and causing magnetic flux lines from adjacent magnets to vary in orientation with respect to one another and with respect to time.

8. The system as in claim 1, wherein at least some of the medial portions of the arms are bowed so that the therapeutic device better approximates a flat surface at the application surface with its combination of magnets and medial portions.

9. The system as claim 1, wherein at least some of the one or more magnet receiving recesses are located on the first and second end portions of the arms.

11. The system as claim 1, wherein at least some of the one or more magnet receiving recesses are located on the medial portions of the arms.

12. The system as in claim 1, wherein the orientation member is a flattened surface on an interior surface of the magnet receiving recess.

13. The system as in claim 1, wherein the orientation member is a keying structure used in combination with the retaining surface to properly orient the magnet.

14. A method for making a therapeutic device having a multitude of magnetic flux lines of different strengths and angular orientation at its application surface, comprising:
inserting a plurality of magnets in a plurality of magnet receiving recesses formed in a plurality of arms such that the magnets are spaced apart from one another at predetermined distances,
orienting each magnet within a magnet receiving recess using an orientation member associated with the magnet receiving recess,
retaining the magnet in the magnet receiving recess in a fixed, predetermined angular orientation with respect to the magnet receiving recess, and
connecting the plurality of arms to one another to form a pattern of magnets, thereby providing at an application surface of a therapeutic device a multitude of magnetic flux lines of different strengths and angular orientations.

15. The method of claim 14, further comprising producing snowflake-like pattern of arms by connecting the arms to one another at a medial portion of each arm piece.

16. The method of claim 14, further comprising a chain-like pattern of arms by connecting the arms to one another end to end.

17. The method of claim 14, wherein the arms are rigid.

18. The method of claim 4, wherein at least some of the arms are flexible so that they may flex when used thereby causing the application surface to bend and causing magnetic flux lines from adjacent magnets to vary with respect to one another and with time.

19. The method of claim 14, further comprising smoothing out the surface at the application surface of the therapeutic device by forming at least some of the medial portions of the arms into a bowed shape.

20. The method as in claim 14, wherein the orientation member is a flattened surface on an interior surface of the magnet receiving recess.

21. The method as in claim 14, wherein the orientation member is a keying structure used in combination with the retaining surface to properly orient the magnet.
